(19) United States
(12) Patent Application Publication
(10) Pub. No.: US 2026/0240540 A1
(43) Pub. Date: Aug. 20, 2026

(54) STAPLING DEVICE WITH FIRING LOCKOUT ASSEMBLY

(71) Applicant: Covidien LP, Mansfield, MA (US)

(72) Inventors: Roanit A. FERNANDES, Bethany, CT (US); Kenneth H. WHITFIELD, North Haven, CT (US)

(21) Appl. No.: 19/162,440

(22) PCT Filed: Mar. 14, 2024
(86) PCT No.: PCT/IB2024/052454

§ 371 Date: Sep. 5, 2025

Related U.S. Application Data
(60) Provisional application No. 63/453,560, filed on Mar. 21, 2023.

(51) Int. Cl. A61B 17/072

(52) U.S. Cl. CPC A61B 17/07207; A61B 2017/07257; A61B 2017/07271; A61B 2017/07278; A61B 2017/07285

(57) ABSTRACT

A surgical stapling device includes a tool assembly having an anvil and a cartridge assembly. The cartridge assembly includes a channel that defines a cavity, and a staple cartridge that is received within the cavity of the channel. The channel includes a side wall that supports a spring arm that extends into the cavity, and the anvil supports at least one lockout member that is engaged with the spring arm to move the lockout member from a locked position to an unlocked position when the staple cartridge is inserted into the cavity of the channel.

This Application claims priority from U.S. Provisional Patent Application 63/453,560, 21 Mar. 2023 filed, the entire content of which is incorporated herein by reference.

### FIELD

This technology is generally related to surgical stapling devices, and more particularly, to surgical stapling devices including lockout assemblies.

### BACKGROUND

Surgical stapling devices configured for endoscopic use are commonly used during surgical procedures to minimize patient trauma and reduce patient recovery times. Typically, endoscopic stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between unclamped and clamped positions in response to movement of the drive assembly from a retracted position to an advanced position. The cartridge assembly includes a staple cartridge that includes a cartridge body that supports staples and an actuation sled that is movable through the cartridge body in response to movement of the drive assembly from the retracted position to the advanced position to eject the staples from the cartridge body.

Some stapling devices include a staple cartridge that can be replaced after each firing of the stapling device to facilitate reuse of the stapling device. To ensure that a stapling device is not fired with a spent staple cartridge, i.e., a staple cartridge having no staples, or without a staple cartridge, some stapling devices include lockouts.

A continuing need exists for a surgical stapling device having a cost-effective assembly for preventing firing of the stapling device when the stapling device includes a spent staple cartridge or an inoperative staple cartridge.

### SUMMARY

This disclosure is directed to a surgical stapling device that includes a tool assembly having an anvil and a cartridge assembly. The cartridge assembly includes a channel that defines a cavity, and a staple cartridge that is received within the cavity of the channel. The channel includes a side wall that supports a spring arm that extends into the cavity, and the anvil supports at least one lockout member that is engaged with the spring arm to move the lockout member from a locked position to an unlocked position when the staple cartridge is inserted into the cavity of the channel.

Aspects of this disclosure are directed to a tool assembly including a cartridge assembly, an anvil, and a first lockout member. The cartridge assembly includes a channel, a spring arm, and a staple cartridge. The channel defines a longitudinal axis and includes a first side wall, a second side wall, and a bottom wall that define a cavity. The bottom wall defines a first knife slot. The spring arm has a distal portion that is secured to the first side wall of the channel and a proximal portion. The spring arm is movable between a non-deformed state in which the proximal portion of the spring arm extends into the cavity of the channel and a deformed state in which the proximal portion of the spring arm is positioned along the first side wall of the channel. The staple cartridge is releasably received within the cavity of the channel and includes a cartridge body, staples, and an actuation sled. The cartridge body defines a second knife slot and staple receiving pockets positioned on opposite sides of the second knife slot. The second knife slot is longitudinally aligned with the first knife slot, and the actuation sled is movable within the cartridge body from a sled retracted position to a sled advanced position to eject the staples from the cartridge body. The anvil is coupled to the cartridge assembly such that the tool assembly is movable between unclamped and clamped positions. The anvil defines a third knife slot. The first lockout member is supported on the anvil and is engaged with the spring arm. The first lockout member is movable, in response to movement of the spring arm from the non-deformed state to the deformed state, between a locked position in which the first lockout member extends at least partially across the third knife slot and an unlocked position in which the first lockout member is spaced from the third knife slot. The staple cartridge is positioned to engage the spring arm when the staple cartridge is inserted into the cavity of the channel to move the spring arm from the non-deformed state to the deformed state.

In aspects of the disclosure, the spring arm includes a body and a finger that is engaged with the first lockout member and extends from the body of the spring arm in a direction transverse to the longitudinal axis of the channel.

In some aspects of the disclosure, the finger defines an elongated slot, and the first lockout member includes a post that is received within the elongated slot of the finger of the spring arm.

In certain aspects of the disclosure, the anvil defines a longitudinal axis and a first channel that extends in a direction transverse to the longitudinal axis of the anvil, and the first lockout member is movable within the first channel between the locked position and the unlocked position.

In aspects of the disclosure, the first channel communicates with the third knife slot.

In some aspects of the disclosure, the cartridge body of the staple cartridge includes a proximal wall that is movable from a non-deformed state to a deformed state to close a proximal end of the second knife slot.

In certain aspects of the disclosure, the spring arm is engaged with the proximal portion of the cartridge body to urge the proximal wall of the cartridge body towards the deformed state.

In aspects of the disclosure, the actuation sled includes a central body portion that is received within the second knife slot, and the central body portion retains the proximal wall of the cartridge body in the non-deformed state when the actuation sled is in the sled retracted position.

In some aspects of the disclosure, the cartridge body of the staple cartridge includes a cutout, and the actuation sled is received within the cutout of the cartridge body when the actuation sled is in the sled retracted position.

In certain aspects of the disclosure, the spring arm is positioned to engage the actuation sled within the cutout when the actuation sled is in the sled retracted position to prevent movement of the spring arm from the deformed state to the non-deformed state.

In aspects of the disclosure, the tool assembly includes a second lockout member that is supported on the anvil, and the second lockout member is positioned distally of the first lockout member and is movable between a locked position in which the second lockout member extends at least partially across the third knife slot and an unlocked position in which the second lockout member is spaced from the third knife slot.

In some aspects of the disclosure, the anvil defines a second channel, and the second lockout member is movable within the second channel from the locked position to the unlocked position.

In some aspects of the disclosure, the tool assembly includes a biasing member to urge the second lockout member towards the locked position.

In certain aspects of the disclosure, the central body portion of the actuation sled supports a knife that is movable with the actuation sled from the sled retracted position to the sled advanced position, and the knife is positioned to engage the second lockout member as the actuation sled moves from the sled retracted position towards the sled advanced position to move the second lockout member from the locked position to the unlocked position.

Other aspects of this disclosure are directed to a surgical stapling device including an adapter assembly, a drive assembly, and a tool assembly. The adapter assembly has a proximal portion and a distal portion, and the tool assembly is supported on the distal portion of the adapter assembly. The drive assembly is at least partially received within the adapter assembly and is movable between a drive retracted position and a drive advanced position. The tool assembly includes a cartridge assembly, an anvil, and a first lockout member. The cartridge assembly includes a channel, a spring arm, and a staple cartridge. The channel defines a longitudinal axis and includes a first side wall, a second side wall, and a bottom wall that define a cavity. The bottom wall defines a first knife slot. The spring arm has a distal portion secured to the first side wall of the channel and a proximal portion. The spring arm is movable between a non-deformed state in which the proximal portion of the spring arm extends into the cavity of the channel and a deformed state in which the proximal portion of the spring arm is positioned along the first side wall of the channel. The staple cartridge is releasably received within the cavity of the channel and includes a cartridge body, staples, and an actuation sled. The cartridge body defines a second knife slot and staple receiving pockets positioned on opposite sides of the second knife slot. The second knife slot is longitudinally aligned with the first knife slot, and the actuation sled is movable within the cartridge body from a sled retracted position to a sled advanced position in response to movement of the drive assembly from the drive retracted position to the drive advanced position to eject the staples from the cartridge body. The anvil is coupled to the cartridge assembly such that the tool assembly is movable between an unclamped position and a clamped position. The anvil defines a third knife slot. The first lockout member is supported on the anvil and is engaged with the spring arm. The first lockout member is movable, in response to movement of the spring arm from the non-deformed state to the deformed state, between a locked position in which the first lockout member extends at least partially across the third knife slot and an unlocked position in which the first lockout member is spaced from the third knife slot. The staple cartridge is positioned to engage the spring arm when the staple cartridge is inserted into the cavity of the channel to move the spring arm from the non-deformed state to the deformed state.

Other aspects of the disclosure are directed to a cartridge assembly including a channel, a spring arm, and a staple cartridge. The channel defines a longitudinal axis and includes a first side wall, a second side wall, and a bottom wall that define a cavity. The bottom wall defines a first knife slot. The spring arm has a distal portion secured to the first side wall of the channel and a proximal portion, and the spring arm is movable between a non-deformed state in which the proximal portion of the spring arm extends into the cavity of the channel and a deformed state in which the proximal portion of the spring arm is positioned along the first side wall of the channel. The staple cartridge is releasably received within the cavity of the channel and includes a cartridge body, staples, and an actuation sled. The cartridge body defines a second knife slot and staple receiving pockets positioned on opposite sides of the second knife slot. The second knife slot is longitudinally aligned with the first knife slot, and the actuation sled is movable within the cartridge body from a sled retracted position to a sled advanced position to eject the staples from the cartridge body. The cartridge body of the staple cartridge includes a proximal wall that is movable from a non-deformed state to a deformed state to close a proximal end of the second knife slot, and the spring arm is engaged with the proximal portion of the cartridge body to urge the proximal wall of the cartridge body towards the deformed state.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the stapling device in an unclamped, pre-fired position;

FIG. 2 is side perspective view of the tool assembly of the stapling device shown in FIG. 1 with a staple cartridge of a cartridge assembly of the tool assembly separated from a channel of the cartridge assembly;

FIG. 3 is an exploded view of the staple cartridge of the cartridge assembly shown in FIG. 2;

FIG. 4 is a perspective view of an anvil assembly of the tool assembly of the stapling device shown in FIG. 1;

FIG. 5 is an enlarged view of the proximal portion of the anvil shown in FIG. 4 with a lockout member separated from the anvil;

FIG. 6 is an enlarged view of the indicated area of detail shown in FIG. 4 illustrating a proximal portion of the anvil;

FIG. 7 is a perspective view from a distal end of the channel of the cartridge assembly shown in FIG. 2;

FIG. 8 is an enlarged perspective view of the indicated area of detail shown in FIG. 7 illustrating a proximal portion of the channel of the cartridge assembly;

FIG. 9 is an enlarged perspective view of the proximal portion of the channel of the cartridge assembly shown in FIG. 2 with a spring arm separated from the channel;

FIG. 10 is a side perspective, cutaway view of a distal portion of a drive assembly of the stapling device shown in FIG. 1;

FIG. 11 is a perspective view from the proximal end of the tool assembly of the stapling device shown in FIG. 2 with the staple cartridge removed and the tool assembly in the unclamped position;

FIG. 12 is a perspective view from the proximal end of the tool assembly of the stapling device shown in FIG. 2 with the staple cartridge removed and the tool assembly in the clamped position;

FIG. 13 is a plan view of the proximal portion of the channel of the cartridge assembly, the lockout member of the anvil, and the distal portion of the drive assembly of the stapling device shown in FIG. 1 with the staple cartridge removed and the lockout member in a locked position;

FIG. 14 is a perspective, cutaway view of the proximal portion of the cartridge assembly and the lockout member of the anvil with the lockout member in an unlocked position;

FIG. 15 is a plan view of the proximal portion of the cartridge assembly and the lockout member of the anvil with the lockout member in the unlocked position;

FIG. 16 is a plan view of the staple cartridge of the cartridge assembly shown in FIG. 2 with an actuation sled in a sled retracted position;

FIG. 17 is a plan view of the staple cartridge of the cartridge assembly shown in FIG. 2 without the actuation sled in the sled retracted position;

FIG. 18 is a plan view of the lockout member of the anvil, the distal portion of the drive assembly, and the staple cartridge of the cartridge assembly of the stapling device shown in FIG. 1 without the actuation sled;

FIG. 19 is an enlarged view of the indicated area of detail shown in FIG. 18;

FIG. 20 is a plan view of the lockout member of the anvil, the distal portion of the drive assembly, and the staple cartridge of the cartridge assembly of the stapling device shown in FIG. 1 with the actuation sled in a sled advanced position;

FIG. 21 is an enlarged view of a proximal portion of an alternate version of the channel of the cartridge assembly of the stapling device shown in FIG. 1;

FIG. 22 is a perspective, cutaway, exploded view of the proximal portion of the channel assembly shown in FIG. 21;

FIG. 23 is a perspective view of a proximal portion of an alternate version of the staple cartridge of the stapling device shown in FIG. 1 with an actuation sled and knife absent from staple cartridge;

FIG. 24 is a perspective view of the proximal portion of the staple cartridge shown in FIG. 23 with the actuation sled and knife shown in phantom;

FIG. 25 is a perspective, cutaway view of the proximal portion of an alternate version of the anvil of the stapling device shown in FIG. 1 with proximal and distal lockout members in locked positions;

FIG. 26 is a perspective view of the proximal portion of the channel of the cartridge assembly shown in FIG. 21 with a spring arm separated from the channel;

FIG. 27 is a plan view of the proximal portion of the channel of the cartridge assembly of the stapling device shown in FIG. 1 and the proximal and distal lockout members in the locked positions;

FIG. 28 is a cross-sectional view taken along section lines 28-28 of FIG. 27;

FIG. 29 is a plan view of the proximal portion of the cartridge assembly shown in FIGS. 26 and 27 with the actuation sled and knife in retracted positions within the staple cartridge and the proximal and distal lockout members in unlocked positions;

FIG. 30 is a cross-sectional view taken along section line 30 -30 of FIG. 29;

FIG. 31 is a plan view of the proximal portion of the cartridge assembly shown in FIG. 29 with the drive assembly in the drive retracted position and the actuation sled in the sled advanced position;

FIG. 32 is an enlarged view of the indicated area of detail shown in FIG. 31; and

FIG. 33 is a plan view of a proximal portion of the cartridge assembly shown in FIG. 29 with the actuation sled in the sled retracted position without the knife absent from the staple cartridge.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes a tool assembly having an anvil and a cartridge assembly. The cartridge assembly includes a channel that defines a cavity, and a staple cartridge that is received within the cavity of the channel. The channel includes a side wall that supports a spring arm that extends into the cavity, and the anvil supports at least one lockout member that is engaged with the spring arm to move the lockout member from a locked position to an unlocked position when the staple cartridge is inserted into the cavity of the channel.

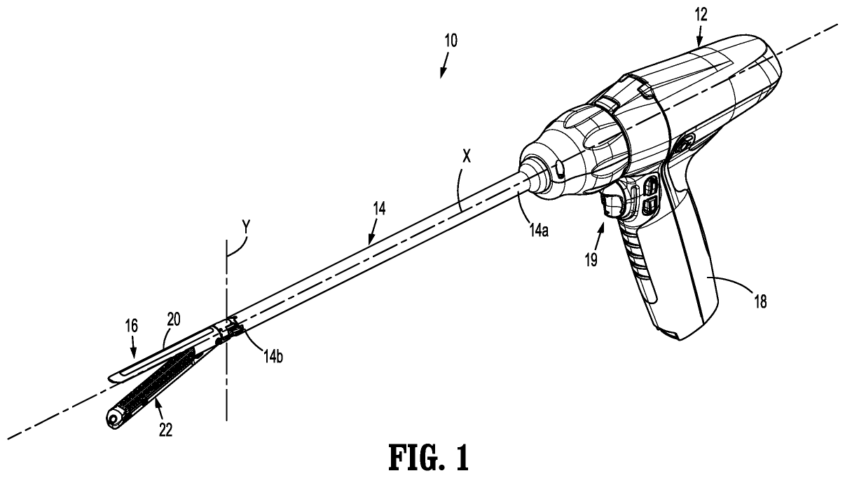

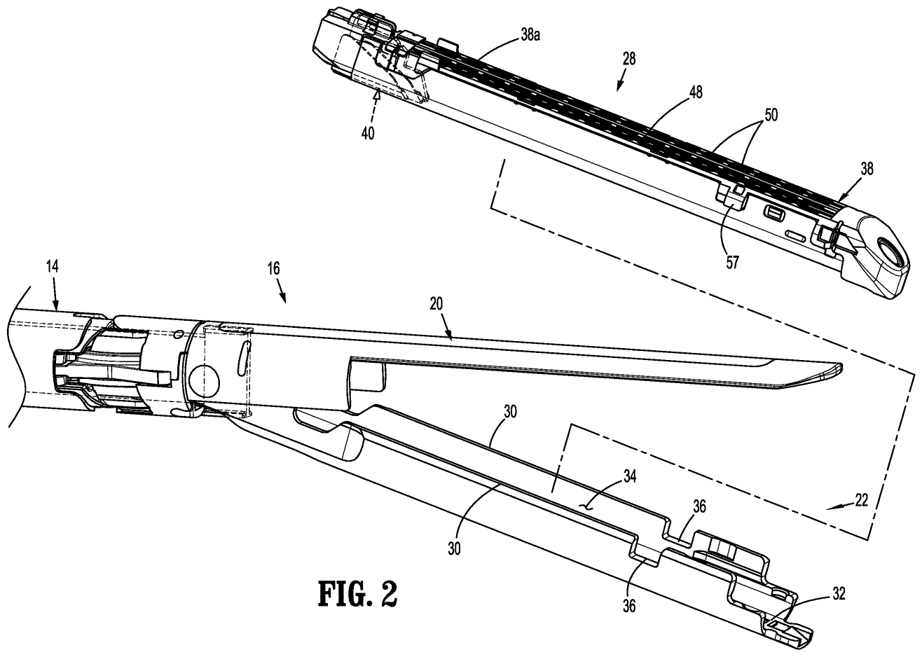

FIGS. 1 and 2 illustrate a surgical stapling device shown generally as stapling device 10 that includes a handle assembly 12, an adapter assembly 14, and a tool assembly 16. The handle assembly 12 is powered and includes a stationary handgrip 18 and an actuation button or buttons 19. The actuation buttons 19 are operable to actuate various functions of the tool assembly 16 via the adapter assembly 14, i.e., approximation of the tool assembly 16, and stapling and cutting of tissue. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices. U.S. Pat. No. 5,865,361 describes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 supports the tool assembly 16. In aspects of the disclosure, the tool assembly 16 is pivotably coupled to the distal portion 14b of the adapter assembly 14 to facilitate articulation of the tool assembly 16 in relation to the adapter assembly 14 about an articulation axis “Y”.

The tool assembly 16 includes an anvil 20 and a cartridge assembly 22. In aspects of the disclosure, the cartridge assembly 22 is pivotably supported in relation to the anvil 20 to facilitate movement of the tool assembly 16 between an unclamped position (FIG. 1) and a clamped position. Alternately, the anvil 20 can be pivotable towards the cartridge assembly 22 to move the tool assembly to the clamped position.

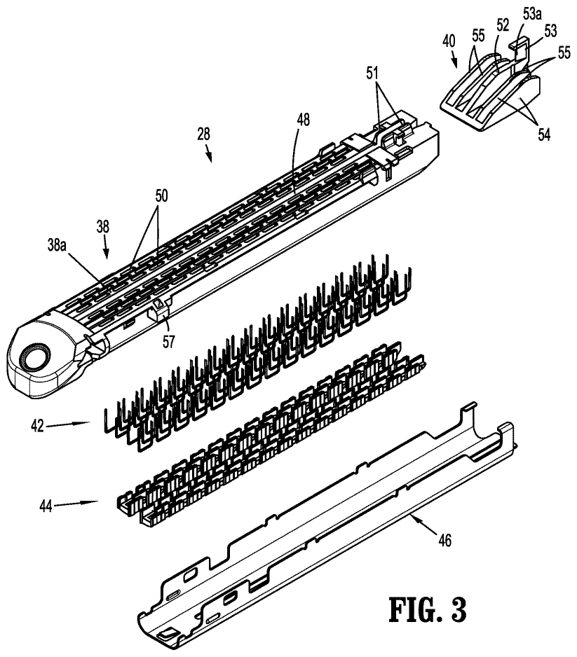

FIGS. 2 and 3 illustrate the cartridge assembly 22 which includes a channel 26 and a staple cartridge 28. The channel 26 includes side walls 30 and a bottom wall 32 that define a cavity 34. The staple cartridge 28 is removably received within the cavity 34 of the channel 26 and is replaceable to facilitate reuse of the stapling device 10. Each of the side walls 30 of the channel 26 has an upper end nearest the anvil 20 that defines a recess 36.

The staple cartridge 28 includes a cartridge body 38, an actuation sled 40, staples 42, pushers 44, and a staple guard 46. The cartridge body 38 defines a knife slot 48 and staple receiving pockets 50 that are positioned on each side of the knife slot 48. The cartridge body 38 includes knife guards 51 that are positioned on opposite sides of the knife slot 48 at a position proximal of the staple receiving pockets 50. In aspects of the disclosure, the staple receiving pockets 50 are aligned in two or more rows on the opposite sides of the knife slot 48. In some aspects of the disclosure, the knife slot 48 is centrally located in the cartridge body 38, although it is envisioned that the knife slot 48 could be offset from the central axis of the cartridge body 38.

The cartridge body 38 includes a tissue engaging surface 38a and laterally extending protrusions 57 (only one is shown) that are received within the recesses 36 (FIG. 2) of the channel 26 to properly position the staple cartridge 28 within the cavity 34 of the channel 26. The staples 42 and the pushers 44 are received within the staple receiving pockets 50 of the cartridge body 38, and the staple guard 46 is secured to the bottom of the cartridge body 38 to retain the staples 42 and pushers 44 within the cartridge body 38. The actuation sled 40 includes a central body portion 52 that supports a knife 53, and cam members 54. The central body portion 52 of the actuation sled 40 and the knife 53 are received within the knife slot 48 of the cartridge body 38 and the cam members 54 are positioned on opposite sides of the central body portion 52 and have angled cam surfaces 55. The cam surfaces 55 of the cam members 54 are positioned to sequentially engage the pushers 44 as the actuation sled 40 moves through the cartridge body 38 from a sled retracted position to a sled advanced position to lift the pushers 44 within the staple receiving pockets 50 and eject the staples 42 from the cartridge body 38. The knife 53 is supported on and extends upwardly from the central body portion 52 of the actuation sled 40 above the tissue engaging surface 38a of the cartridge body 38 towards the anvil 20 to cut tissue clamped between the staple cartridge 28 and the anvil 20.

The knife 53 is positioned within the knife slot 48 of the cartridge body 38 and includes a distally facing cutting edge 53a that extends between a gap defined between the anvil 20 and the cartridge assembly 22 when the tool assembly 16 is in the clamped position. When the actuation sled 40 is in the sled retracted position, the knife 53 is positioned between the knife guards 51.

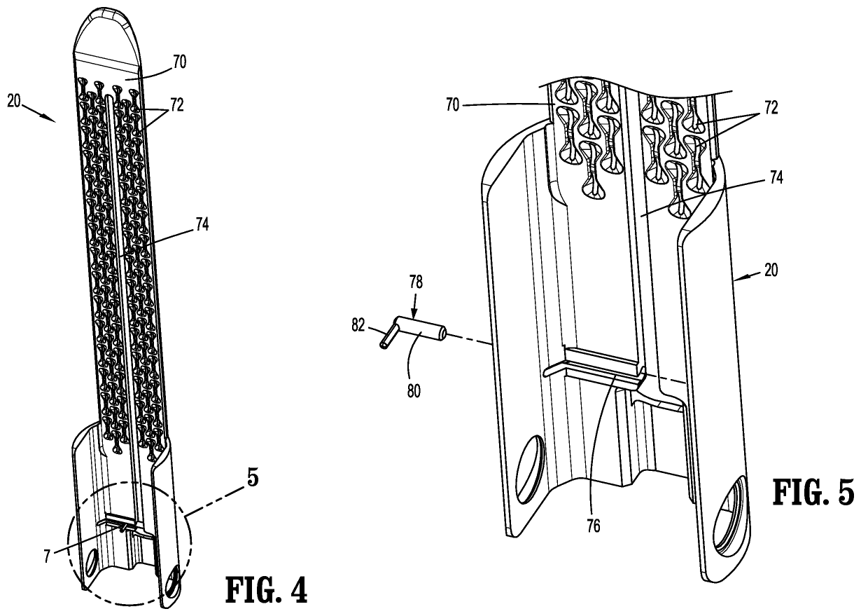

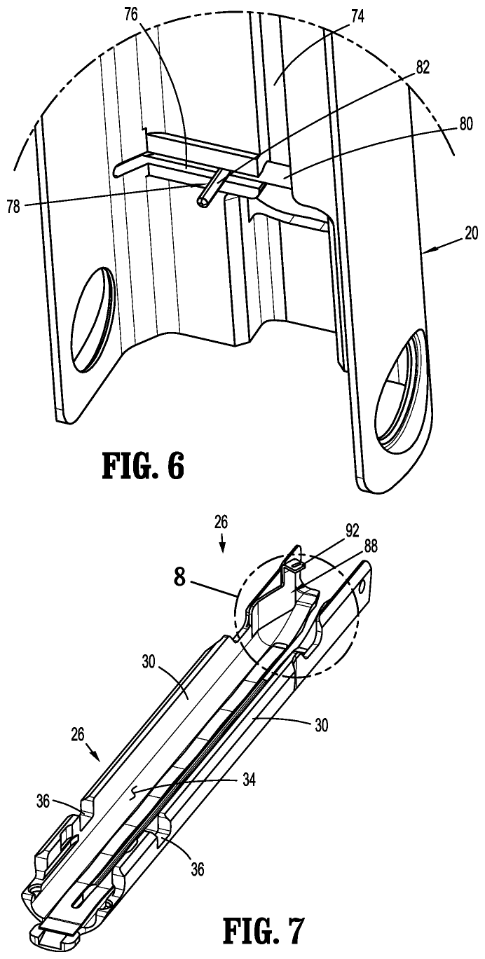

FIGS. 4-6 illustrate the anvil 20 of the stapling device 10 which includes a tissue engaging surface 70 that defines staple forming pockets 72, and a knife slot 74. The anvil 20 defines a transverse channel 76 that is positioned adjacent to and communicates with the knife slot 74 of the anvil 20. The knife slot 74 of the anvil 20 is longitudinally aligned with the knife slot 48 of the cartridge body 38 when the tool assembly 16 (FIG. 1) is in the clamped position.

The anvil 20 supports a lockout member 78 that includes a body 80 and a post 82 that extends upwardly from the body 80 towards the cartridge assembly 22. The body 80 of the lockout member 78 is received within the transverse channel 76 of the anvil 20 and is movable between an unlocked position in which the body 80 of the lockout member 78 is spaced outwardly of the knife slot 74 of the anvil 20, and a locked position in which the body 80 of the lockout member 78 extends across the knife slot 74 of the anvil 20.

FIGS. 7-9 illustrate the channel 26 of the cartridge assembly 22 which includes the side walls 30 and the bottom wall 32 that define the cavity 34. The bottom wall 32 of the channel 26 defines a knife slot 84 that is longitudinally aligned with the knife slot 48 in the cartridge body 38 of the staple cartridge 28 when the staple cartridge 28 is received within the cavity 34 of the channel 26. The proximal portion of one of the side walls 30 of the channel 26, the supporting side wall 30, supports a spring arm 88 that includes a resilient body 90 and a transversely extending finger 92 that extends towards the opposite side wall 30 of the channel 26. The finger 92 defines an elongated slot 94 that receives the post 82 of the lockout member 78. In aspects of the disclosure, the spring arm 88 has a distal portion 88a that is secured to the side wall 30 of the channel 26 and a proximal portion 88b that is angled or positioned inwardly from the supporting side wall 30 into the cavity 34 of the channel 26. In some aspects of the disclosure, the supporting side wall 30 of the channel 26 defines a recess 96 that has a depth and a configuration to receive the spring arm 88. When the spring arm 88 is deformed outwardly from the staple cartridge 38 as described in detail below, the spring arm 88 is received within the recess 96.

FIG. 10 illustrates a distal portion of the drive assembly 66 of the stapling device 10 (FIG. 1) which includes a flexible drive beam 102 and a working member 104. The flexible drive beam 102 has a distal portion that is secured to a proximal portion of the working member 104. The working member 104 has an I-shaped configuration and includes a first beam 106, a second beam 108, and a vertical strut 110. The vertical strut 110 has a first end that is connected to the first beam 106 and a second end that is secured to the second beam 108. The drive assembly 66 is movable between a drive retracted position, through a drive clamped position, and a drive advanced position. When the drive assembly 66 is moved from the drive retracted position to the drive clamped position, the first beam 106 engages the anvil 20 and the second beam 108 engages the channel 26 to move the tool assembly 16 (FIG. 1) from the unclamped position to the clamped position. When the drive assembly 66 moves from the drive clamped position to the drive advanced position, a distal portion of the vertical strut of the working member 104 of the drive assembly 66 abuts the actuation sled 40 to advance the actuation sled 40 from the sled retracted position to the sled advanced position to eject the staples 42 from the cartridge body 38 and cut tissue clamped between the anvil 20 and the cartridge assembly 22. When the stapling device 10 is fired and the drive assembly 66 is moved from the drive advanced position to the drive retracted position, the actuation sled 40 remains in the sled advanced position.

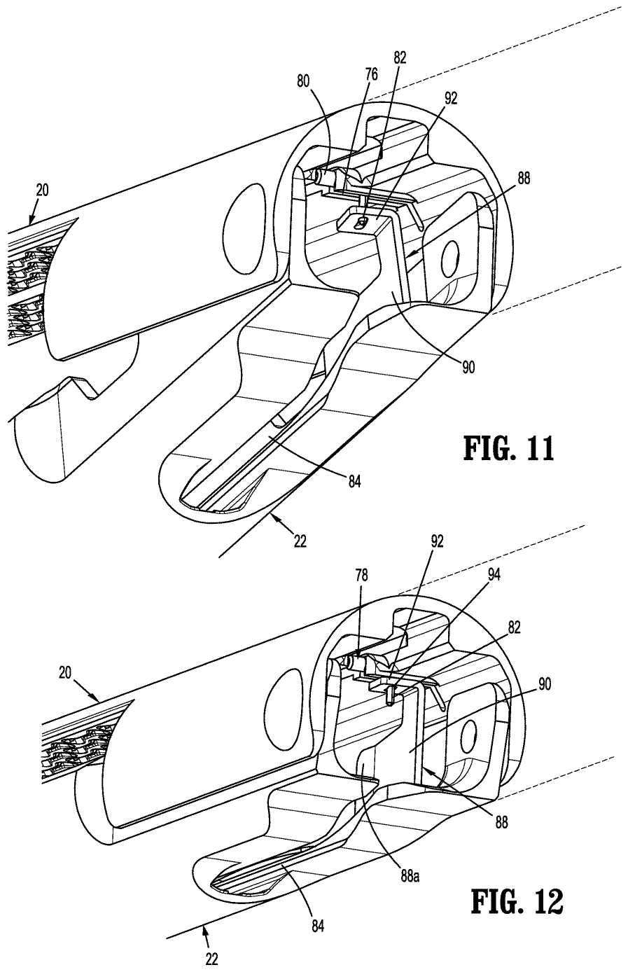

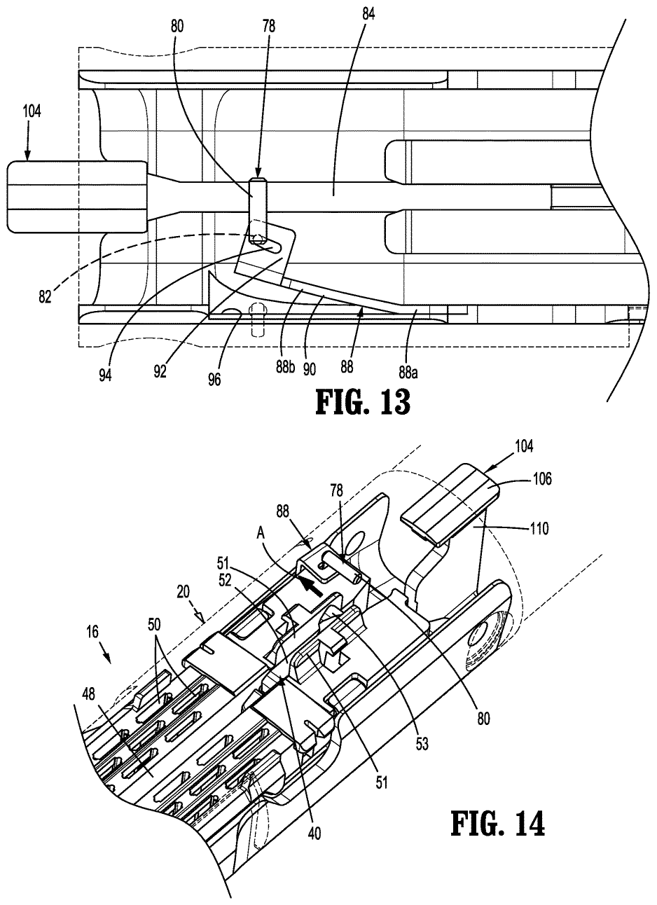

FIGS. 11-13 illustrate a proximal portion of the tool assembly 16 prior to insertion of the staple cartridge 38 into the cavity 34 of the channel 26. Prior to insertion of the staple cartridge 38 into the cavity 34 of the channel 26, the post 82 of the lockout member 78 is received in the slot 94 on the finger 92 of the spring arm 88. The distal portion 88a of the spring arm 88 is in a non-deformed state and projects into the cavity 34 of the channel 26 to retain the lockout member 78 in the locked position. In the locked position, the body 80 of the lockout member 78 extends across the knife slot 84 of the channel 26 of the cartridge assembly 22 (FIG. 2) to prevent movement of the drive assembly 66 from the drive retracted position to the drive advanced position.

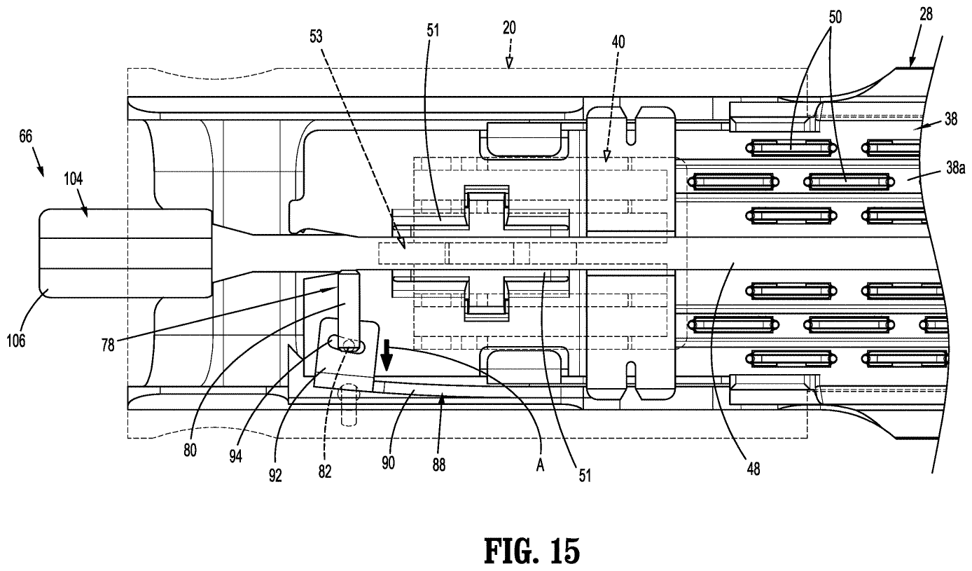

FIGS. 14 and 15 illustrate the cartridge assembly 22 after the staple cartridge 28 is inserted into the cavity 34 of the channel 26 of the cartridge assembly 22. When the staple cartridge 28 is inserted into the cavity 34 of the channel 26 of the cartridge assembly 22, the cartridge body 38 engages the spring arm 88 and moves the spring arm 88 outwardly in the direction of arrows “A” from the non-deformed state to the deformed state towards the supporting side wall 30 of the channel 26. When the staple cartridge 28 is received within the cavity 34 of the channel 26 of the cartridge assembly 22, the spring arm 88 is received within the recess 96 in the supporting side wall 30 of the channel 26 and the lockout member 78 is moved to the unlocked position in which the body 80 of the lockout member 78 is spaced outwardly of the knife slot 84 in the channel 26. In this position of the lockout member 78, the drive assembly 66 is unobstructed by the lockout member 78 and can move from the drive retracted position to the drive advanced position. The lockout member 78 prevents advancement of the drive assembly 66 when a staple cartridge 28 is not loaded into the channel 26 of the cartridge assembly 22. The lockout member 78 does not prevent advancement of the drive assembly 66 when the staple cartridge 28 is spent, i.e., the staple cartridge has been fired and does not include staples.

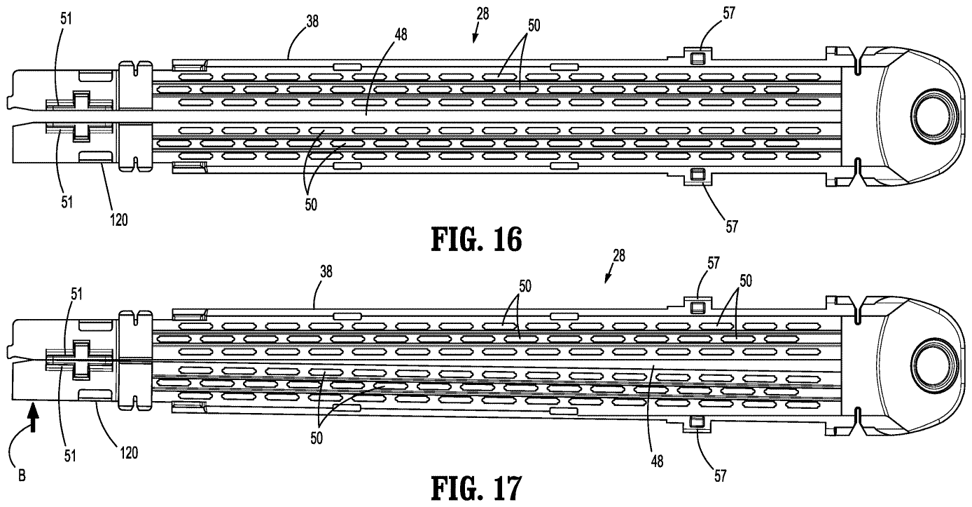

FIGS. 16 and 17 illustrate the staple cartridge 28 of the stapling device 10 without an actuation sled 40 in the sled retracted position. In aspects of the disclosure, a proximal wall 120 of the cartridge body 38 of the staple cartridge 28 is deformable inwardly in the direction of arrow “B” in FIG. 17 from a non-deformed state to a deformed state upon application of a predetermined force to the proximal wall 120 of the cartridge body 38. When the proximal wall 120 is moved to the deformed state, a proximal portion of the knife slot 48 is closed to prevent passage of the working member 104 of the drive assembly 66 from the drive retracted position into the knife slot 48. When the actuation sled 40 is positioned in the sled retracted position, the central body portion 52 of the actuation sled 40 prevents the proximal portion of the cartridge body 38 from moving to the deformed state.

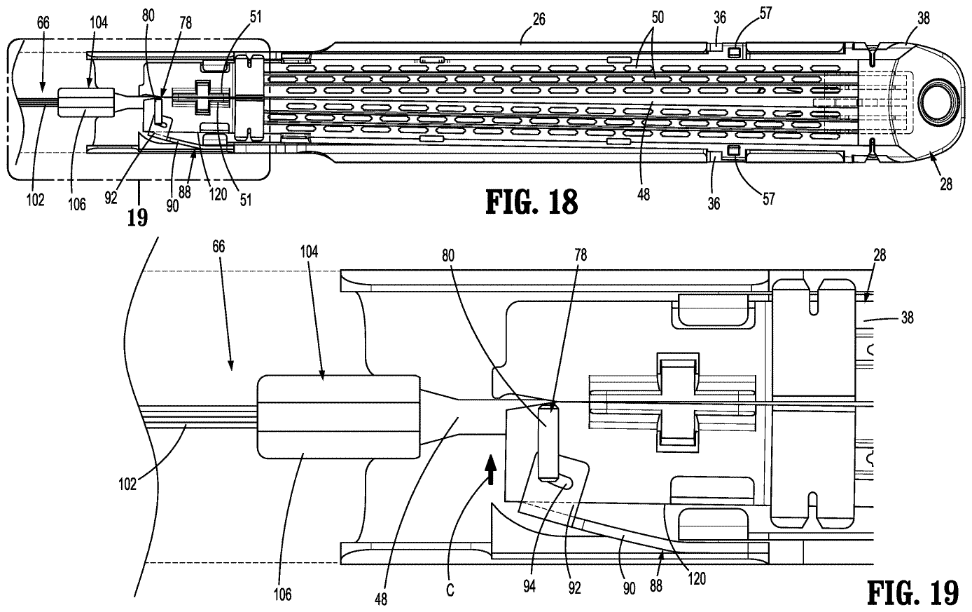

FIGS. 18 and 19 illustrate the staple cartridge 28 positioned within the cavity 34 of the channel 26 of the cartridge assembly 22 after the stapling device 10 (FIG. 1) is fired with the drive assembly 66 returned to the drive retracted position. As illustrated, after the stapling device 10 is fired, the actuation sled 40 remains in the distal portion of the cartridge body 38. When the actuation sled 40 is not positioned in the proximal portion of the knife slot 48 of the cartridge body 38 of the staple cartridge 28, the spring arm 88 presses on the proximal wall 120 of the cartridge body 38 to move the proximal wall 120 of the cartridge body 38 from the non-deformed state to the deformed state to close the proximal end of the knife slot 48 of the cartridge body 38 of the staple cartridge 28. As the proximal wall 120 of the cartridge body 38 moves from the non-deformed state to the deformed state, the spring arm 88 moves inwardly in the direction of arrow “C” in FIG. 19 to return the lockout member 78 back to the locked position in which the body 80 of the lockout member 78 extends across the knife slot 48. The deformable proximal wall 120 of the cartridge body 38 of the staple cartridge 28 in association with the spring arm 88 and lockout member 78 prevent advancement of the drive assembly 66 with a spent staple cartridge 28.

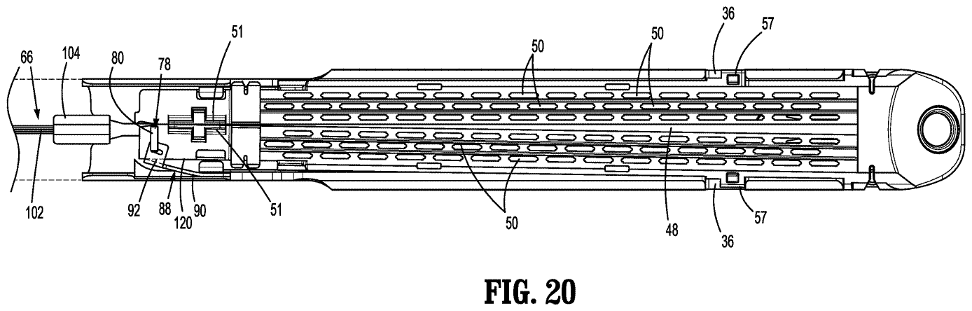

FIG. 20 illustrates a defective staple cartridge 38 that does not include an actuation sled 40. When a defective staple cartridge 38 is inserted into the channel 26 of the cartridge assembly 22 prior to firing of the stapling device 10 (FIG. 1), the spring arm 88 moves the proximal wall 120 of the cartridge body 38 from the non-deformed state to the deformed state to close the proximal end of the knife slot 48 of the cartridge body 38 of the staple cartridge 28. As described above regarding post firing, as the proximal wall 120 of the cartridge body 38 moves from the non-deformed state to the deformed state, the spring arm 88 moves inwardly to return the lockout member 78 back to the locked position in which the body 80 of the lockout member 78 extends across the knife slot 48. The deformable proximal wall 120 of the cartridge body 38 of the staple cartridge 28 in association with the spring arm 88 and lockout member 78 prevent advancement of the drive assembly 66 when the staple cartridge 28 is defective and does not include an actuation sled 40.

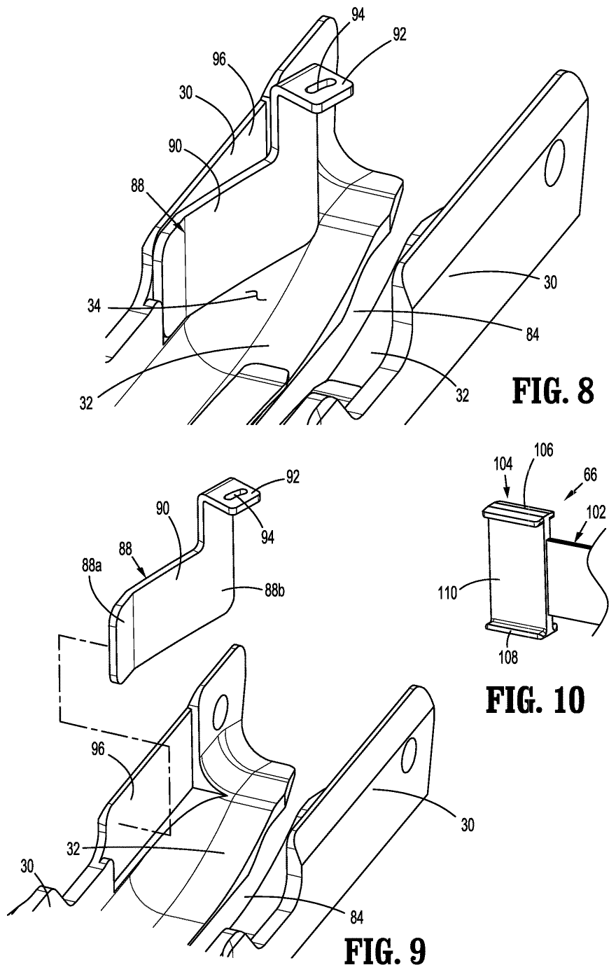

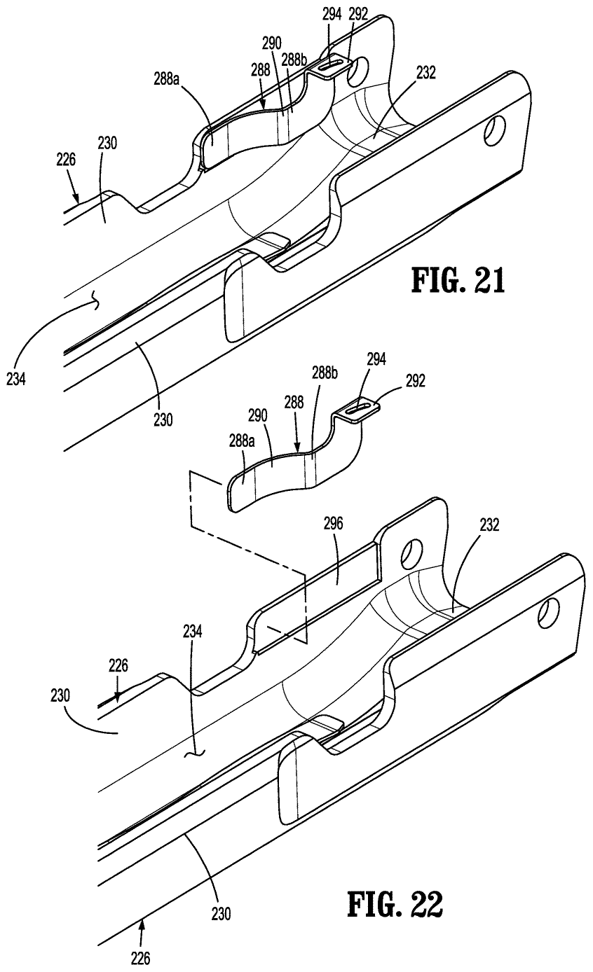

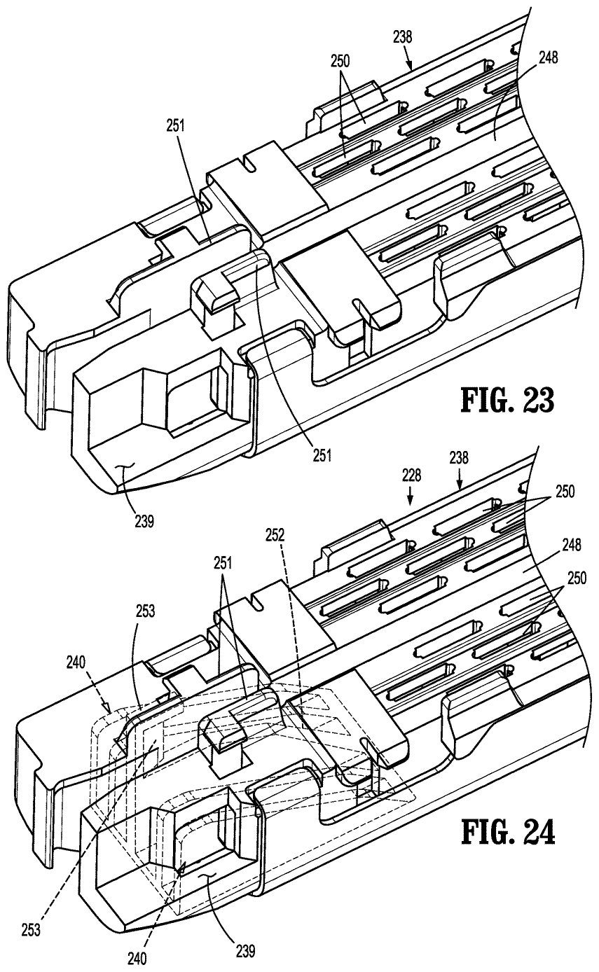

FIGS. 21-33 illustrate an alternate version of the tool assembly of the stapling device 10 shown generally as tool assembly 216 (FIG. 30). The tool assembly 216 includes an anvil 220 (FIG. 26) and a cartridge assembly 222 (FIG. 30). FIGS. 21-24 illustrate the cartridge assembly 222 which is substantially like the cartridge assembly 22 and includes a channel 226 (FIGS. 21 and 22) and a staple cartridge 228 (FIGS. 23 and 24). Like the channel 26 (FIG. 8), the channel 226 includes side walls 230 and a bottom wall 232 that define a cavity 234. The staple cartridge 228 is removably received within the cavity 234 of the channel 226 and is replaceable to facilitate reuse of the stapling device 10 (FIG. 1). The bottom wall 232 of the channel 226 defines a knife slot 284.

The proximal portion of one of the side walls 230 of the channel 226, the supporting side wall 230, supports a spring arm 288 that includes a resilient body 290 and a transversely extending finger 292 that extends towards the side wall 230 of the channel 226 opposite to the supporting side wall 230. The finger 292 defines an elongated slot 294. In aspects of the disclosure, the spring arm 288 has a distal portion 288a that is secured to the side wall 230 of the channel 226 and a proximal portion 288b that is angled or positioned inwardly of the supporting side wall 230 within the cavity 234 of the channel 226. In some aspects of the disclosure, the supporting side wall 230 of the channel 226 defines a recess 296 (FIG. 22) that has a depth and a configuration to receive the spring arm 288.

The staple cartridge 228 (FIG. 23) of the cartridge assembly 222 is like the staple cartridge 28 (FIG. 3) and includes a cartridge body 238, an actuation sled 240, staples (not shown), pushers (not shown), and a staple guard (not shown). A knife 253 is supported on and extends upwardly from the central body portion 252 of the actuation sled 240 to a position above the tissue engaging surface 238a of the cartridge body 328 towards the anvil 220 to cut tissue clamped between the staple cartridge 228 and the anvil 220. In alternative version of the stapling device 10 (not shown), the knife 253 can be coupled to the actuation sled 240 and movable between lowered and raised positions.

The cartridge body 238 defines a knife slot 248 and staple receiving pockets 250 that are positioned on opposite sides of the knife slot 248. The cartridge body 238 includes knife guards 251 that are positioned on opposite sides of the knife slot 248 at a position proximally of the staple receiving pockets 250. As described above regarding knife guards 51 (FIG. 3), the knife guards 251 shield the knife 253 when the actuation sled 240 is in the sled retracted position. The staple cartridge 228 differs from the staple cartridge 28 (FIG. 3) in that the cartridge body 238 of the staple cartridge 228 is substantially rigid and does not include a deformable proximal wall, and in that the proximal portion of the cartridge body 238 defines a cutout 239 (FIG. 24) to expose the actuation sled 240 when the actuation sled 238 in in the sled retracted position. When the actuation sled 240 is in the sled retracted position, the spring arm 288 engages the actuation sled 240 as the staple cartridge 228 is positioned within the cavity 234 of the channel 226 of the cartridge assembly 222 to move the spring arm 288 to the deformed state and the first lockout member 278a to the unlocked position.

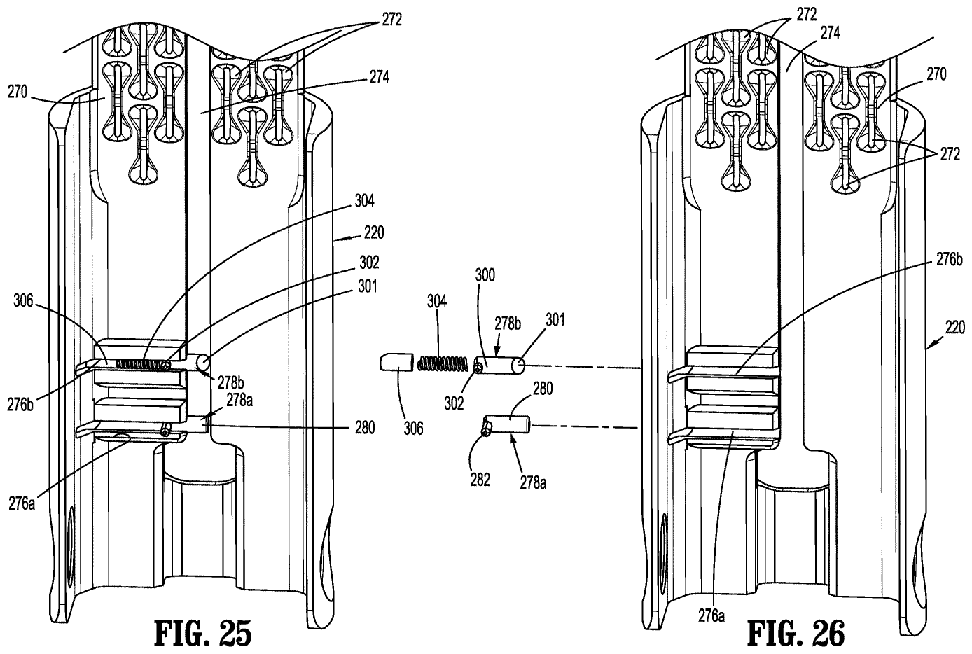

FIGS. 25-28 illustrate the anvil 220 which includes a tissue engaging surface 270 that defines staple forming pockets 272 and a knife slot 274. The anvil 220 defines a first transverse channel 276a that is positioned adjacent to the knife slot 274 of the anvil 220 and communicates with the knife slot 274. The anvil 220 also defines a second transverse slot 276b that is positioned distally of the first transverse channel 276a and communicates with the knife slot 274. The knife slot 274 of the anvil 220 is longitudinally aligned with the knife slot 248 of the cartridge body 238 when the tool assembly 216 (FIG. 1) is in the clamped position.

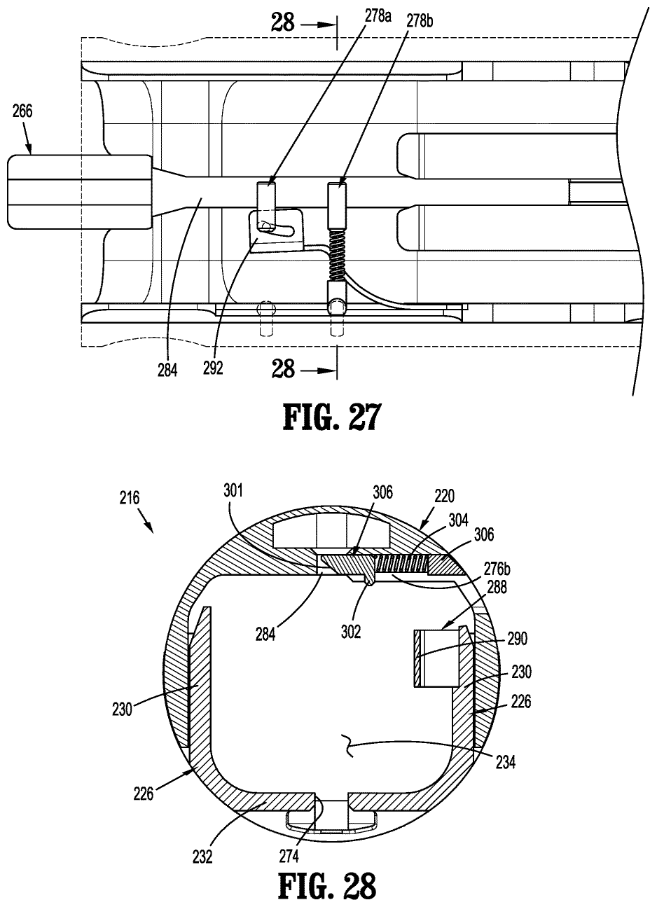

The anvil 220 supports a first lockout member 278a and a second lockout member 278b. The first lockout member 278a includes a body 280 and a post 282 that extends from the body 280 towards the cartridge assembly 222. The body 280 of the lockout member 278 is received within the transverse channel 276a of the anvil 220 and is movable between an unlocked position in which the body 280 of the lockout member 278a is spaced outwardly of the knife slot 274 of the anvil 220 and a locked position in which the body 280 of the lockout member 278a extends across the knife slot 274 of the anvil 220. The first lockout member 278a is engaged with the spring arm 288 as described above regarding lockout member 78 (FIG. 5) and functions identically to the lockout member 78 to prevent advancement of the drive assembly 266 (FIG. 27) when the staple cartridge 228 is not present within the cavity 234 of the channel 226. As such, the first lockout member 278 will not be described in further detail herein.

The second lockout member 278b is received within the second transverse channel 276b of the anvil 220 and includes a body 300 having a first end that is positioned within or adjacent the knife slot 274 in the anvil 220. The first end of the body 300 of the second lockout member 278b includes an angled surface 301. The body 300 includes a protrusion 302 that extends through the transverse channel 276b in the anvil 220 towards the channel 226 of the cartridge assembly 222 to prevent rotation of the body 300 of the second lockout member 278b within the second transverse channel 276b. The second lockout member 278b is movable within the second transverse channel 276b between a locked position in which the body 300 of the second lockout member 278b extends into the knife slot 274 of the anvil 220 and an unlocked position in which the body 300 of the second lockout member 278b is moved outwardly of the knife slot 274 of the anvil 220. In aspects of the disclosure, the second lockout member 278b is urged towards the locked position by a biasing member, e.g., a coil spring 304 that is positioned between a second end of the second lockout member 278b and a plug 306 secured to the anvil 220.

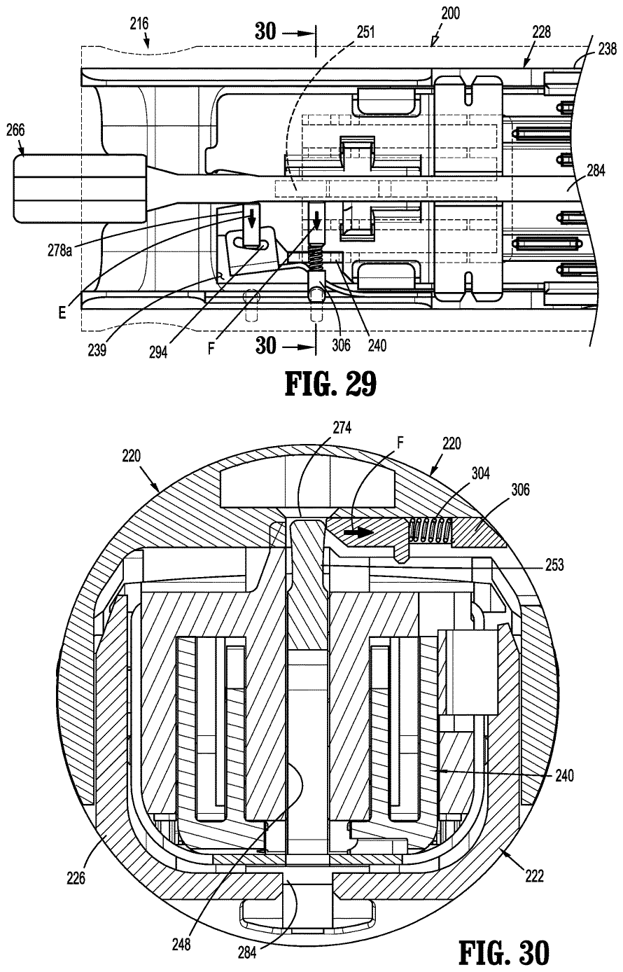

FIGS. 29 and 30 illustrate the tool assembly 216 of the stapling device 10 with the tool assembly in the clamped position prior to firing of the stapling device 10. When the staple cartridge 228 is inserted into the cavity 234 of the channel 226, the exposed portion of the actuation sled 240 of the staple cartridge 228 that is positioned within the cutout 239 of the cartridge body 238 engages the spring arm 288 to move the spring arm 288 to the deformed state to move the first lockout member 278a in the direction of arrow “E” in FIG. 29 from the locked position to the unlocked position spaced outwardly of the knife slot 284 in the channel 226. As described above, in the unlocked position of the first lockout member 278a, the drive assembly 266 can move from the drive clamped position past the first lockout member 278a towards the drive advanced position to actuate the tool assembly 216.

As the drive assembly 266 moves from the drive clamped position towards the drive advanced position past the first lockout member 278a, the knife 253 of the actuation sled 240 engages the second lockout member 278b and moves the second lockout member 278b in the direction of arrow “F” in FIG. 20 from the locked position to the unlocked position. In the unlocked position of the second lockout member 278b, the drive assembly 266 can continue to move towards the drive advanced position to move the actuation sled 240 towards the sled advanced position to actuate the tool assembly 216. In aspects of the disclosure, the knife 253 includes an angled or tapered side wall that engages one end of the body 300 of the second lockout member 278b to urge the second lockout member 278b towards the unlocked position.

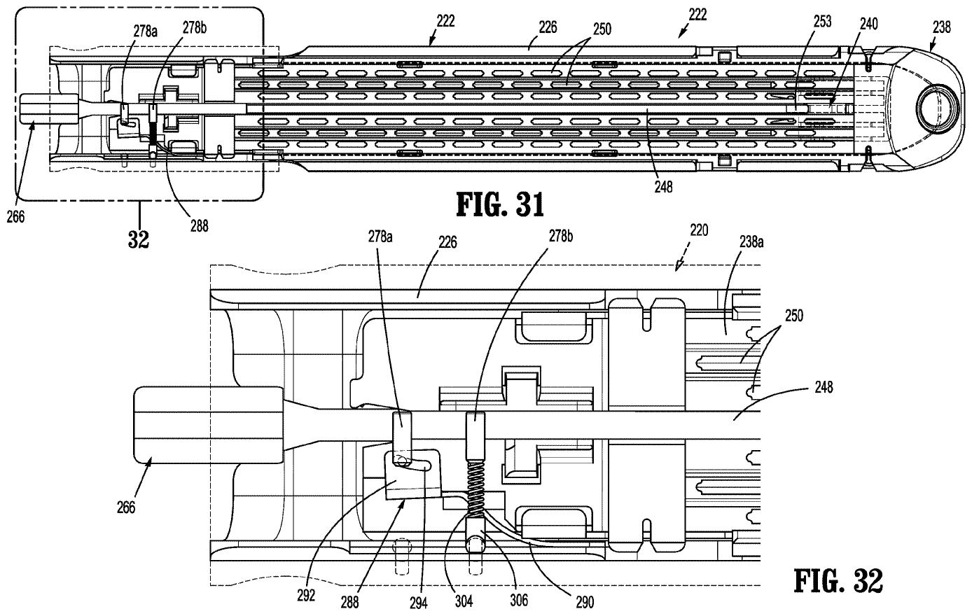

FIGS. 31 and 32 illustrate the tool assembly 216 after the tool assembly 216 has been fired such that the staple cartridge 228 does not include an actuation sled 240 in the sled retracted position. When the actuation sled 240 is not positioned within the cartridge body 240 of the staple cartridge 228 in the sled retracted position, the actuation sled 240 is not positioned (in the cutout 239 of the cartridge body 238) to deform the spring arm 288. Thus, the spring arm 288 remains in the non-deformed state to retain the first locking member 278a in the locked position. In the locked position, the first locking member 278a prevents advancement of the drive assembly 266 from the drive retracted position to the drive advanced position. Similarly, when the knife 253 of the actuation sled 240 is not positioned to engage the second locking member 278b, (such as when the actuation sled 240 and knife 253 are not in the sled retracted position), the spring 304 moves the second lockout member 278b to the locked position. The first and second lockout members 278a, 278b prevent advancement of the drive assembly 266 to the drive advanced position when the actuation sled 240 and knife 253 are not positioned in the sled retracted position.

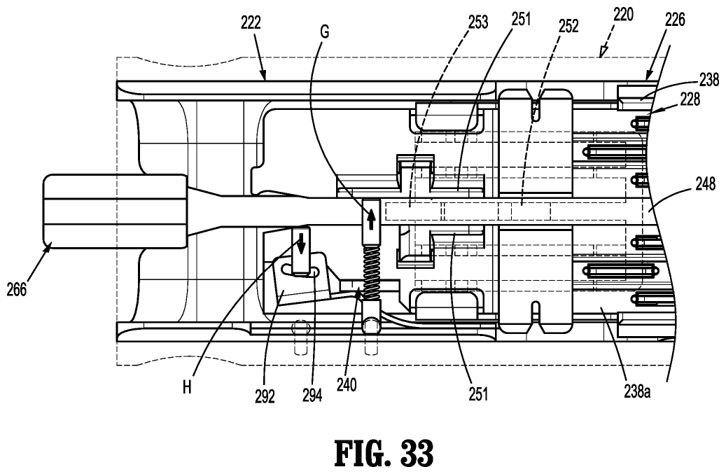

FIG. 33 illustrates the tool assembly 216 of the stapling device 10 (FIG. 1) with a staple cartridge 228 having an actuation sled 240 without a knife 253. When a knife 253 is not included on the actuation sled 240, even when the actuation sled 240 is in the sled retracted position, the second lockout member 278b is moved in the direction indicated by arrow “G” to the locked position to prevent the drive assembly 266 from moving from the drive clamped position to the drive advanced position. As described above, when the actuation sled 240 is in the sled retracted position and the staple cartridge 228 is inserted into the cavity 234 of the channel 226, the first lockout member 278a is moved in the direction of arrow “H” to the unlocked position.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A tool assembly comprising:
a cartridge assembly including:
a channel defining a longitudinal axis and including a first side wall, a second side wall, and a bottom wall, the first side wall, the second side wall, and the bottom wall defining a cavity, the bottom wall defining a first knife slot;
a spring arm having a distal portion secured to the first side wall of the channel and a proximal portion, the spring arm movable between a non-deformed state in which the proximal portion of the spring arm extends into the cavity of the channel and a deformed state in which the proximal portion of the spring arm is positioned along the first side wall of the channel; and
a staple cartridge releasably received within the cavity of the channel, the staple cartridge including a cartridge body, staples, and an actuation sled, the cartridge body defining a second knife slot and staple receiving pockets positioned on opposite sides of the second knife slot, the second knife slot longitudinally aligned with the first knife slot, the actuation sled movable within the cartridge body from a sled retracted position to a sled advanced position to eject the staples from the cartridge body;
an anvil coupled to the cartridge assembly such that the tool assembly is movable between unclamped and clamped positions, the anvil defining a third knife slot; and
a first lockout member supported on the anvil and engaged with the spring arm, the first lockout member movable, in response to movement of the spring arm from the non-deformed state to the deformed state, between a locked position in which the first lockout member extends at least partially across the third knife slot and an unlocked position in which the first lockout member is spaced from the third knife slot, wherein the staple cartridge is positioned to engage the spring arm when the staple cartridge is inserted into the cavity of the channel to move the spring arm from the non-deformed state to the deformed state.

2. The tool assembly of claim 1, wherein the spring arm includes a body and a finger that is engaged with the first lockout member and extends from the body of the spring arm in a direction transverse to the longitudinal axis of the channel.

3. The tool assembly of claim 2, wherein the finger defines an elongated slot, and the first lockout member includes a post that is received within the elongated slot of the finger of the spring arm.

4. The tool assembly of claim 1, wherein the anvil defines a longitudinal axis and a first channel that extends in a direction transverse to the longitudinal axis of the anvil, the first lockout member movable within the first channel between the locked position and the unlocked position.

5. The tool assembly of claim 4, wherein the first channel communicates with the third knife slot.

6. The tool assembly of claim 1, wherein the cartridge body of the staple cartridge includes a proximal wall that is movable from a non-deformed state to a deformed state to close a proximal end of the second knife slot.

7. The tool assembly of claim 6, wherein the spring arm is engaged with the proximal portion of the cartridge body to urge the proximal wall of the cartridge body towards the deformed state.

8. The tool assembly of claim 7, wherein the actuation sled includes a central body portion that is received within the second knife slot, the central body portion retaining the proximal wall of the cartridge body in the non-deformed state when the actuation sled is in the sled retracted position.

9. The tool assembly of claim 1, wherein the cartridge body of the staple cartridge includes a cutout, and the actuation sled is received within the cutout of the cartridge body when the actuation sled is in the sled retracted position.

10. The tool assembly of claim 9, wherein the spring arm is positioned to engage the actuation sled within the cutout when the actuation sled is in the sled retracted position to prevent movement of the spring arm from the deformed state to the non-deformed state.

11. The tool assembly of claim 4, further including a second lockout member supported on the anvil, the second lockout member positioned distally of the first lockout member and movable between a locked position in which the second lockout member extends at least partially across the third knife slot and an unlocked position in which the second lockout member is spaced from the third knife slot.

12. The tool assembly of claim 11, further including a biasing member, wherein the anvil defines a second channel, and the second lockout member is movable within the second channel from the locked position to the unlocked position, the biasing member urging the second lockout member towards the locked position.

13. The tool assembly of claim 12, wherein the central body portion of the actuation sled supports a knife that is movable with the actuation sled from the sled retracted position to the sled advanced position, the knife positioned to engage the second lockout member as the actuation sled moves from the sled retracted position towards the sled advanced position to move the second lockout member from the locked position to the unlocked position.

14. A surgical stapling device comprising:
an adapter assembly having a proximal portion and a distal portion;
a drive assembly at least partially received within the adapter assembly, the drive assembly movable between a drive retracted position and a drive advanced position; and
a tool assembly supported on the distal portion of the adapter assembly, the tool assembly including:
a cartridge assembly including:
a channel defining a longitudinal axis and including a first side wall, a second side wall, and a bottom wall, the first side wall, the second side wall, and the bottom wall defining a cavity, the bottom wall defining a first knife slot;
a spring arm having a distal portion secured to the first side wall of the channel and a proximal portion, the spring arm movable between a non-deformed state in which the proximal portion of the spring arm extends into the cavity of the channel, and a deformed state in which the proximal portion of the spring arm is positioned along the first side wall of the channel; and
a staple cartridge releasably received within the cavity of the channel, the staple cartridge including a cartridge body, staples, and an actuation sled, the cartridge body defining a second knife slot and staple receiving pockets positioned on opposite sides of the second knife slot, the second knife slot longitudinally aligned with the first knife slot, the actuation sled movable within the cartridge body from a sled retracted position to a sled advanced position in response to movement of the drive assembly from the drive retracted position to the drive advanced position to eject the staples from the cartridge body;
an anvil coupled to the cartridge assembly such that the tool assembly is movable between unclamped and clamped positions, the anvil defining a third knife slot; and
a first lockout member supported on the anvil and engaged with the spring arm, the first lockout member movable, in response to movement of the spring arm from the non-deformed state to the deformed state, between a locked position in which the first lockout member extends at least partially across the third knife slot and an unlocked position in which the first lockout member is spaced from the third knife slot, wherein the staple cartridge is positioned to engage the spring arm when the staple cartridge is inserted into the cavity of the channel to move the spring arm from the non-deformed state to the deformed state.

15. The surgical stapling device of claim 14, wherein the spring arm includes a body and a finger that defines an elongated slot and extends from the body of the spring arm in a direction transverse to the longitudinal axis of the channel, and the first lockout member includes a post that is received within the elongated slot of the finger of the spring arm.

16. The surgical stapling device of claim 14, wherein the cartridge body of the staple cartridge includes a proximal wall that is movable from a non-deformed state to a deformed state to close a proximal end of the second knife slot, and the spring arm is engaged with the proximal portion of the cartridge body to urge the proximal wall of the cartridge body towards the deformed state.

17. The surgical stapling device of claim 16, wherein the actuation sled includes a central body portion that is received within the second knife slot, the central body portion retaining the proximal wall of the cartridge body in the non-deformed state when the actuation sled is in the sled retracted position.

18. The surgical stapling device of claim 14, wherein the cartridge body of the staple cartridge includes a cutout, and the actuation sled is received within the cutout of the cartridge body when the actuation sled is in the sled retracted position, the spring arm positioned to engage the actuation sled within the cutout when the actuation sled is in the sled retracted position to prevent movement of the spring arm from the deformed state to the non-deformed state.

19. The surgical stapling device of claim 14, wherein anvil of the tool assembly supports a second lockout member that is positioned distally of the first lockout member, the second lockout member received within a second channel of the anvil and movable between a locked position in which the second lockout member extends at least partially across the third knife slot and an unlocked position in which the second lockout member is spaced from the third knife slot, and the central body portion of the actuation sled supports a knife that is movable with the actuation sled from the sled retracted position to the sled advanced position, the knife positioned to engage the second lockout member as the actuation sled moves from the sled retracted position towards the sled advanced position to move the second lockout member from the locked position to the unlocked position.

20. A cartridge assembly comprising:
a channel defining a longitudinal axis and including a first side wall, a second side wall, and a bottom wall, the first side wall, the second side wall, and the bottom wall defining a cavity, the bottom wall defining a first knife slot;
a spring arm having a distal portion secured to the first side wall of the channel and a proximal portion, the spring arm movable between a non-deformed state in which the proximal portion of the spring arm extends into the cavity of the channel and a deformed state in which the proximal portion of the spring arm is positioned along the first side wall of the channel; and
a staple cartridge releasably received within the cavity of the channel, the staple cartridge including a cartridge body, staples, and an actuation sled, the cartridge body defining a second knife slot and staple receiving pockets positioned on opposite sides of the second knife slot, the second knife slot longitudinally aligned with the first knife slot, the actuation sled movable within the cartridge body from a sled retracted position to a sled advanced position to eject the staples from the cartridge body,
wherein the cartridge body of the staple cartridge includes a proximal wall that is movable from a non-deformed state to a deformed state to close a proximal end of the second knife slot, and the spring arm is engaged with the proximal portion of the cartridge body to urge the proximal wall of the cartridge body towards the deformed state.
